(19) United States
(12) Patent Application Publication
(10) Pub. No.: US 2024/0041658 A1
(43) Pub. Date: Feb. 8, 2024

(54) Perforated Adhesive Laminate

(71) Applicant: ADVANCED SILICONE COATING, Pusignan (FR)

(72) Inventor: Jean-Francois LECOEUVRE, Sorbier (FR)

(73) Assignee: ADVANCED SILICONE COATING, Pusignan (FR)

(21) Appl. No.: 18/259,004

(22) PCT Filed: Dec. 22, 2021
(86) PCT No.: PCT/EP2021/087221

§ 371 Date: Jun. 22, 2023

(30) Foreign Application Priority Data
Dec. 23, 2020 (EP) 20217128.6

(51) Int. Cl. A61F 13/02

(52) U.S. Cl. CPC A61F 13/0233; A61F 13/0253

(57) ABSTRACT

The invention relates to new laminate showing excellent properties when being adhered to a surface such as the skin. The laminate can for example be used for the preparation of articles such as wound dressings which are used for promoting/supporting the process of the healing of a wound.

The invention relates to new laminate showing excellent properties when being adhered to a surface such as the skin. The laminate can for example be used for the preparation of articles such as wound dressings which are used for promoting/supporting the process of the healing of a wound.

A wound can be regarded as the separation of the contiguity of tissues of the skin, wherein this can be combined with loss of substance.

The healing of wounds is based on the ability of the skin to regenerate tissue such as epithelial tissue, connective and supporting tissue. The regeneration of tissue is a complex occurrence of cell activities overlapping with each other, wherein said cell activities promote the healing process step by step. A suitable wound dressing may help to provide a favorable environment for said healing process.

Such wound dressings have to be applied on a surface such as a wound or the skin surrounding said wound. On the one hand, it is essential that the wound dressing adheres well to said surface to prevent an undesired slipping or even removal of the wound dressing. On the other hand, it is also important to ensure that, once a wound dressing has to be removed, for example to change the dressing, the removal of the dressing does not negatively affect the surface (of the skin). In fact, the properties of the layer facing the wound appear to be essential to ensure the above-indicated adherence properties of a wound dressing.

It is reported that perforated layers may have improved properties with regard to the specific adherence requirements of the layer facing the wound. For example, US 2009/0093779 A1 describes a supple thermoplastic section of material comprising a first smooth surface; a second surface facing away from the first smooth surface and a plurality of three-dimensional perforations whose walls extend from the first smooth surface and end in an overhang with a free edge, thus making the second surface rough to touch. Said material, however, seems to be improvable with regard to the peeling properties, such as the strength of the peel force and peeling condition, once peeling (removal) is desired.

Thus, there is still a need for an adhesive material or structure which can, for example, be used for the manufacture of a wound dressing which, once applied to the skin and/or wound, remains there until its secure removal.

Hence, it was an object of the present invention to overcome the above drawbacks.

In particular, it was an object of the present invention to provide a structure which can for example be used in a wound dressing which is capable of ensuring a strength of the peel force that ensures continuance at the place the wound dressing has been applied to. In other words, it is an object of the present invention to provide a structure that prevents the wound dressing for example from slipping or even removal at an undesired time. Further, once removal is desired, the structure should also ensure safe removal without negatively affecting the surface of the skin or wound to which it has been applied.

### SUMMARY

The present invention has unexpectedly solved the above objectives by a new perforated adhesive laminate comprising a first layer comprising a medically acceptable adhesive and a substrate, wherein the laminate has perforation holes which opening has a specific area and wherein said perforation holes form a specific perforation pattern.

Thus, the subject of the present invention is a perforated adhesive laminate comprising
(a) a first layer comprising a medically acceptable adhesive,
(b) a substrate
(c) optionally a second layer comprising a medically acceptable adhesive,
wherein the first layer is coated on one side of the substrate and wherein the optional second layer is coated on the opposite side of the substrate, and

wherein the laminate has perforation holes forming a perforation pattern such that the strength of the first layer's average peel force in machine-direction and the strength of the first layer's average peel force in cross-direction differ no more than 5%,
and/or the amplitude between the maximum and minimum peaks of the peel force curve are no more than 25% of the average peel force; and
wherein the perforation holes have a surface size of between 1.75 to 12.5 mm².

Another aspect of the invention is an assembly comprising the perforated adhesive laminate according to the present invention and a textile or a polymer-based film.

Finally, the invention is directed to the use of the perforated adhesive laminate according to the present invention or the assembly according to the present invention as a wound contact layer and/or backing layer of a wound dressing.

### DETAILED DESCRIPTION OF THE INVENTION

The present perforated adhesive laminate comprises (a) a first layer comprising a medically acceptable adhesive. An adhesive can be regarded as a substance which, when applied to one or both surfaces of two distinct subjects, binds them together and resists their separation. The adhesive comprised in the first layer is medically acceptable. A medically acceptable adhesive is an adhesive which substantially does not negatively affect the environment/surface of the body to which it may be applied. Thus, when for example applied over a wound, the medically acceptable adhesive does not release inadvertently or grow together with the wound tissue, since both can be detrimental to wound healing.

The medically acceptable adhesive comprised in the first layer can be any medically acceptable adhesive known to the skilled person. Examples of medically acceptable adhesives are acrylate-based adhesives, epoxide-based adhesives, urethane-based adhesives and silicone-based adhesives. It is preferred that the medically acceptable adhesive comprised in the first layer is an acrylate-based adhesive or a silicone-based adhesive. More preferred, the medically acceptable adhesive comprised in the first layer is a silicon-based adhesive.

In a preferred embodiment the first layer has a thickness from 20 μm to 225 μm, more preferably from 30 μm to 215 μm, in particular from 40 μm to 200 μm.

Preferably the first layer has a thickness of about 45 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm or about 190 μm.

In a further preferred embodiment, the first layer (a) comprises a medically acceptable silicone-based adhesive and has a thickness of 20 μm to 225 μm, more preferably from 40 μm to 200 μm.

In a preferred embodiment the first layer consists of a medically acceptable adhesive.

Further, the present perforated adhesive laminate comprises (b) a substrate. In line with the present application a substrate can be considered as a carrier comprising an inert material. Inert materials suitable for a substrate are known in the art. Non-limiting examples of suitable inert materials are polymers, such as polyethylene-based materials, polypropylene-based materials, polyester-based materials, such as materials from polyethylene terephthalate (PET), polyamide-based materials, polyvinylchloride-based materials, polyacrylate-based materials, polymethacrylate-based materials, polyurethane-based materials, such as materials from polyester urethane or polyether urethane, silicone-based materials and mixtures thereof. In a preferred embodiment the substrate comprises a polyurethane-based material.

Further, the substrate can have any form as long as the first layer can be coated on one side of the substrate. Preferably the substrate is a film.

In a preferred embodiment the substrate has a thickness of 5μm to 50 μm, more preferably 10 μm to 40 μm, in particular 20 μm to 30 μm.

Preferably the substrate has a thickness of about 21 μm, about 22 μm, about 23 μm, about 24 μm, about 25 μm, about 26 μm, about 27 μm, about 28 μm or about 29 μm, especially about 25 μm.

In a further preferred embodiment, the substrate comprises polyurethane-based material, preferably in form of a film and has a thickness of 5μm to 50 μm, more preferably from 10 μm to 40 μm.

First layer (a) is applied on substrate (b). In a preferred embodiment, the laminate is construed such that the first layer (a) is the wound contact layer. In other words, the first layer is arranged such that the side opposite to the side applied on the substrate is facing the wound.

The laminate according to the present invention comprises (c) an optional second layer comprising a medically acceptable adhesive. As far as the medically acceptable adhesive comprised in optional second layer (c) is concerned, generally the same applies as described above with regard to the medically acceptable adhesive comprised in the first layer.

Thus, the medically acceptable adhesive comprised by optional second layer (c) can for example be acrylate-based adhesives, epoxide-based adhesives, urethane-based adhesives or silicone-based adhesives.

The medically acceptable adhesive comprised by optional second layer (c) can preferably be the same adhesive as the medically acceptable adhesive comprised by first layer (a).

Alternatively preferred, the medically acceptable adhesive comprised by optional second layer (c) can be another adhesive as the medically acceptable adhesive comprised by first layer (a).

It is preferred that the medically acceptable adhesive comprised in the optional second layer is an acrylate-based adhesive or a silicone-based adhesive. More preferred, the medically acceptable adhesive comprised in the optional second layer is an acrylate-based adhesive.

In a preferred embodiment the optional second layer consists of a medically acceptable adhesive.

In a preferred embodiment optional second layer (c) layer has a thickness of 5μm to 100 μm, more preferably 10 μm to 70 μm, in particular 20 μm to 40 μm, especially about 30 μm.

Preferably the optional second layer has a thickness of about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm or about 39 μm.

In a further preferred embodiment, optional second layer (c) comprises a medically acceptable acrylate-based adhesive and has a thickness of 5μm to 100 μm, in particular 20 μm to 40 μm.

In a preferred embodiment optional second layer (c) is applied onto the opposite side of the substrate onto which the first layer is applied. Thus, optional second layer (c) is optionally applied onto the wound-remote side of substrate (b).

The laminate according to the invention has perforation holes forming a perforation pattern such that the strength of the first layer's average peel force in machine-direction and the strength of the first layer's average peel force in cross-direction differ no more than 5%, preferably no more than 4%, in particular no more than 3%.

A perforation hole can be considered as an opening going through the perforated laminate, i.e. completely through first layer (a) and substrate (b) as well as optional second layer (c). Perforation holes can be made by any device suitable for the perforation of a laminate such as nails, needles or a punch. Alternatively, perforation can be carried out by die cutting, kiss cutting and by ultrasonic cutting.

The opening of a perforation hole can be any form. In other words, the form of the opening of a perforation hole can for example be rectangular, square, circular, elliptic, triangular, pentagonal, hexagonal, octagonal or diamond-shaped. In a preferred embodiment the openings of the perforation holes are square, circular or elliptic, preferably circular.

The openings of the perforation holes have a size of between 1.75 mm² and 12.5 mm², preferably between 2.0 mm² and 8.5 mm², in particular between 2.25 mm² and 5.0 mm 2 .

In case that the perforation hole is circular, the opening size of a perforation hole between 1.75 mm² and 12.5 mm² substantially corresponds to a diameter of the opening hole of about 1.5 mm to about 3.9 mm. Correspondingly, again if circular, the opening size of a perforation hole between 2.25 mm² and 5.0 mm² substantially corresponds to a diameter of the opening hole of about 1.7 mm to about 2.5 mm.

The perforation holes in the laminate form a perforation pattern such that the strength of the first layer's average peel force in machine-direction and the strength of the first layer's average peel force in cross-direction differ no more than 5%, and/or the amplitude between the maximum and minimum peaks of the peel force curve is no more than 25%, preferably no more than 15% of the average peel force.

In line with the present application the laminate can be considered to have a machine direction and a cross direction. Generally, by the process of manufacturing, such as drawing a laminate in machine-direction, said laminate can get desired properties, such as advantageous shrink properties. The cross-direction is generally a direction being inclined by a certain angle from the machine direction. Said angle is usually 0° to 90° C.

The strength of the average peel force in machine-direction and the average strength of the peel force in cross-direction and the amplitude between the maximum and minimum peaks of the peel force curve are determined as explained in the experimental section below.

By forming the perforation pattern such that the strength of the first layer's average peel force in machine-direction and the strength of the first layer's average peel force in cross-direction differ no more than 5%, both the strength of the first layer's average peel force in machine-direction and the strength of the first layer's average peel force in cross-direction are substantially the same and thus, the known peeling off from one direction or side of the laminate can preferably be prevented or at least significantly reduced. Thus, advantageous homogenous strengths of the average peel forces of the first layer in both directions of the whole laminate can be achieved. Similar considerations apply in case that the amplitude between the maximum and minimum peaks of the peel force curve is no more than 25% of the average peel force. Again, an advantageous homogenous peel force of the first layer and thus of the whole laminate can be achieved and peeling off the side of the laminate can be preferably prevented or at least significantly reduced.

Depending on the distance of the perforation holes from each other, the corresponding angle of distribution of said holes and the optional alignment to the machine-direction/cross direction, there are different perforation patterns.

A perforation pattern with perforation holes is known, wherein the perforation holes are arranged at equidistance from each other to form an equilateral triangle (angle 60°) aligned with machine direction and cross direction is known. Such a perforation pattern can be seen in FIG. 1. Due to the pattern, poor balance of the strengths of the average peel forces in machine direction and cross direction can be achieved. Further, the pattern leads to a disadvantageously high amplitude between the maximum and minimum peaks of the peel force curve.

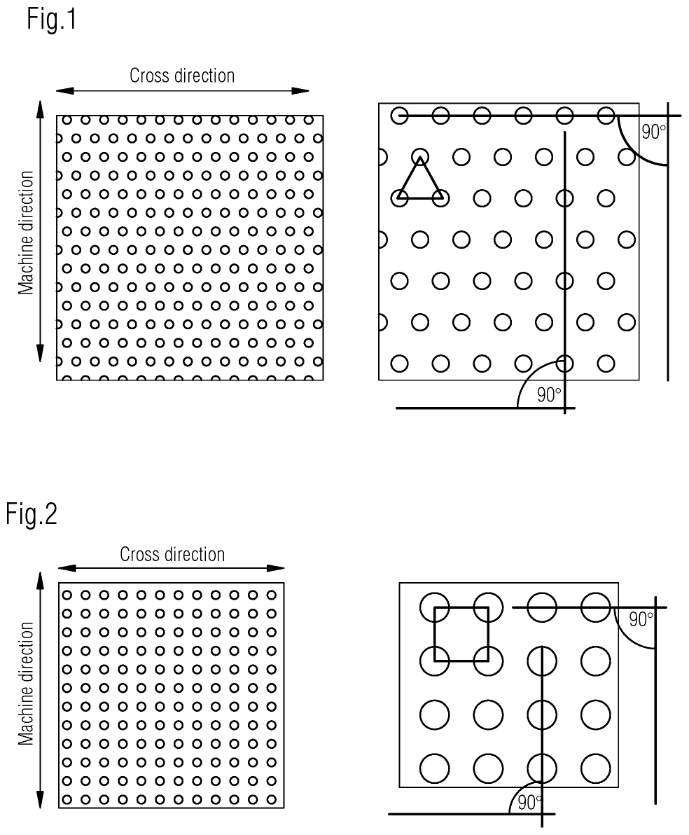

In a preferred embodiment of the invention the perforation holes are arranged at equidistance from each other to form a square aligned to machine direction and cross direction. Such a perforation pattern can be seen in FIG. 2. Due to the pattern, a good balance of the strengths of the average peel forces in machine direction and cross direction can be achieved. Further, the pattern results in a high amplitude between the maximum and minimum peaks of the peel force curve.

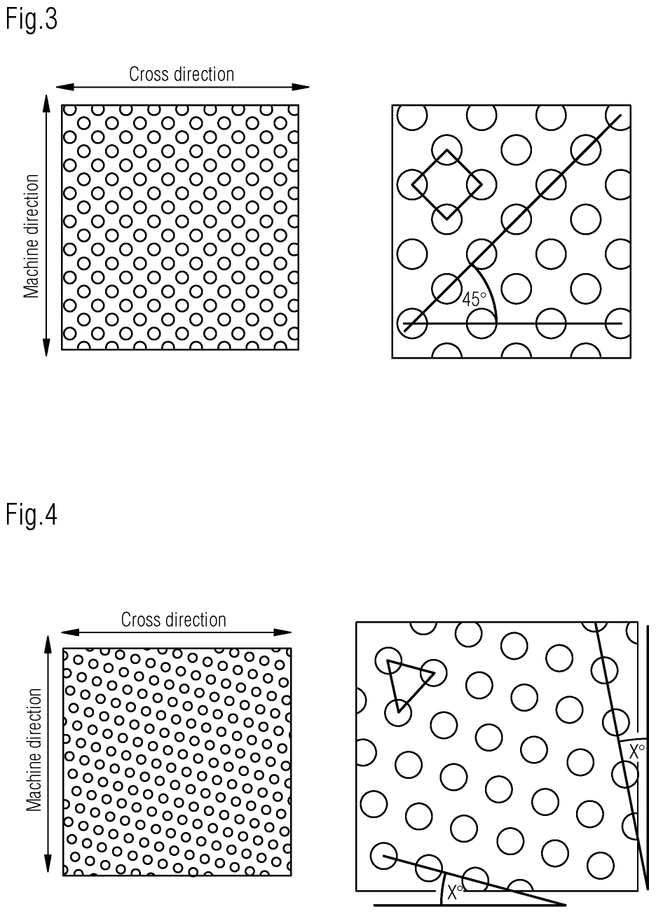

In a preferred embodiment of the invention the perforation holes are arranged at equidistance from each other to form a square at a 45° angle from machine direction and cross direction. Such a perforation pattern can be seen in FIG. 3. Due to the pattern, a good balance of the strengths of the average peel forces in machine direction and cross direction can be achieved. Further, the pattern results in a high amplitude between the maximum and minimum peaks of the peel force curve.

In a more preferred embodiment of the invention the perforation holes are arranged at equidistance from each other to form an equilateral triangle (angle 60°), wherein the aligned holes constitute an angle of between 10° and 20°, preferably between 12° and 18°, more preferably between 13° and 17°, in particular about 15° from machine direction and cross direction. Such a perforation pattern with 15° from the machine and cross direction can be seen in FIG. 4. Due to the pattern, a good balance of the strengths of the average peel forces in machine direction and cross direction can be achieved. Further, since there is no alternance between perforated rows and plain rows at the front of the peel direction, the pattern leads to an advantageously low amplitude between the maximum and minimum peaks of the peel force curve.

In a preferred embodiment of the invention the sum of the areas of the opening of the perforated holes is between 10% and 50%, preferably between 12% and 45%, more preferably between 15% and 30% of the total surface of the laminate. These areas can also be referred to as open (or opened) areas of the laminate due to the absence of any material in this area.

The sum of the areas of the opening of the perforated holes is preferably about 15%, about 16%, about 17%, about 18%, about 19%, about 20%, about 21%, about 22%, about 23%, about 24%, about 25%, about 26%, about 27%, about 28%, about 29% or about 30%, more preferably about 25%.

It turned out that through a high open area rate the present laminate exhibits good adherence to a surface such as the skin.

In a preferred embodiment of the invention a liner is applied to the first layer comprising a medically acceptable adhesive.

A liner can be regarded as a releasable/removable layer which can be applied to a layer with adhesive properties such as the present first layer. Thus, when applied and as long as not removed from the adhesive layer, a liner can prevent said layer with adhesive properties from being adhered to an undesired surface or object. In other words, a liner can be regarded as releasable layer which protects the laminate from being adhered to an undesired surface and/or being polluted/contaminated before being adhered to the desired surface.

Materials for liners are well known in the art. A liner can for example be a paper liner. Alternatively, the liner can be a polymeric liner. Suitable polymers for polymeric liners are for example polyurethane, polyether urethane, polyester urethane, polyester, polypropylene, polyethylene, polyamide, polyvinylchloride, polyorganosiloxane (silicone), polyacrylate, polymethacrylate or mixtures thereof, preferably polyethylene.

In a preferred embodiment of the present invention the present perforated adhesive laminate comprises (a) a first layer comprising a medically acceptable adhesive, (b) a substrate and (c) a second layer comprising a medically acceptable adhesive, wherein the first layer is applied on one side of the substrate and wherein the optional second layer is applied on the opposite side of the substrate; wherein a liner is applied to either the first or the second layer comprising a medically acceptable adhesive.

In an alternative preferred embodiment of the present invention the present perforated adhesive laminate comprises (a) a first layer comprising a medically acceptable adhesive, (b) a substrate and (c) a second layer comprising a medically acceptable adhesive, wherein the first layer is applied on one side of the substrate and wherein the optional second layer is applied on the opposite side of the substrate; wherein liners are applied to the first as well as the second layer comprising a medically acceptable adhesive. In case that liners are applied to the first and the second layer comprising a medically acceptable adhesive, respectively, it is preferred that the liner applied to the first layer is a polymeric liner, in particular a polyethylene liner, and that the liner applied to the second layer is a paper liner.

During production of the perforated laminate it may also be necessary to remove a liner and replace it by a new liner.

In a further aspect the invention relates to a method for the preparation of the perforated adhesive laminate according to the invention, wherein the method comprises the steps of:
(i) providing a laminate comprising (a) a first layer comprising a medically acceptable adhesive, (b) a substrate, (c) optionally a second layer comprising a medically acceptable adhesive,
   wherein the first layer is applied on one side of the substrate and wherein the optional second layer is applied on the opposite side of the substrate, and wherein optionally a liner is applied to the first and/or the second layer comprising a medically acceptable adhesive
(ii) die cutting the laminate provided by step (i), wherein die cutting is preferably performed such that the perforation holes are arranged at equidistance from each other to form an equilateral triangle, wherein the aligned holes constitute an angle of between 10° and 20° from machine direction and cross direction.

As far as conditions, compounds, properties etc. of the first layer, the substrate, the optional second layer and the liner(s) in step (i) are concerned, the same applies as described with regard to the perforated laminate according to the invention.

The laminate provided in step (i) can for example be obtained by laminating (a) a first layer comprising a medically acceptable adhesive, (b) a substrate, (c) optionally a second layer comprising a medically acceptable adhesive in the defined order and optionally applying liner(s) to the first and/or second layer comprising a medically acceptable adhesive, respectively.

In step (ii) the laminate provided by step (i) is die cut such that preferably at least a partial perforation of the laminate is obtained. Die cutting can be carried out by every suitable punching apparatus known in the art, wherein the die (punch) has to be selected such that the openings of the perforation holes have an area of between 1.75 mm² and 12.5 mm². Die cutting can for example be carried out as full layer punching or stamping perforation with male and female parts. The die cutting process can be carried out as a continuous process with rotary tools. The rotary die cutting unit comprises a cylindrical rotary cutting tool (called die) facing a counter flat cylinder, which can be also referred to as male and female cylinders. The material to be die cut passes through both cylinders. Alternatively, die cutting can be carried out as a discontinuous process on a linear machine, i.e. punching a part of the object then moving forward the object and punching the next part etc. The depth of the protuberant part (blade) of the die depends on the thickness of the material to be cut. Depending on (micro) gaps between the blade and counter cylinder, the product is fully perforated or not. Further, die cutting is preferably performed such that the perforation holes are preferably arranged at equidistance from each other to form an equilateral triangle, wherein the aligned holes constitute an angle of between 10° and 20°, in particular about 15°, from machine direction and cross direction.

Further, the invention relates to an assembly comprising the perforated adhesive laminate according to the invention and a textile or a polymer-based film.

In line with this application a textile can be referred to as a flexible material which can be prepared by weaving, knitting, crocheting, knotting, tatting, felting, bonding or braiding of yarns or threads and thereby creating an interlocking network of these yarns or threads. These yarns or threads in turn can be obtained by spinning raw fibres from either natural or synthetic sources into long and twisted lengths. Examples of textiles are woven fabrics, non-woven fabrics, knittings, nettings or braids, tulle or felt.

In a preferred embodiment of the assembly of the invention, the textile is a woven fabric or a non-woven fabric.

A woven fabric is a fabric made by interlacing two or more threads at right angles to one another and can for example be obtained by weaving. Woven fabrics can be produced on a loom and made of many threads woven on a warp and a weft.

A non-woven fabric is a fabric-like material which can be made from staple fibre (short) and long fibres (continuous long), bonded together by chemical, mechanical, heat or solvent treatment.

In a preferred embodiment of the invention the woven fabric or the non-woven fabric is based on natural or synthetic fibers.

Natural fibres can be referred to as fibres having a natural origin such as plants, animals or minerals. Natural fibres generally can be used without having undergone a chemical transformation reaction. Examples are silk, cellulose, cotton and wool.

Synthetic fibres can be referred to as fibres which have been either chemically synthesized or natural fibres which have been chemically transformed or modified.

Examples are viscose fibres or fibres made from polymers such as poly(meth)acrylate, polyamide, polyimide, polyamidimide, polyurethane, polyester such as polyethylene terephthalate and polybutylene terephthalate, polyether, polyacrylnitrile, polyalkylene such as polyethylene and polypropylene.

In a preferred embodiment the polymer-based film comprised by the assembly has a solubility in water of less than 10 mg/l, more preferably less than 1 mg/ml, in particular from 0,0001 to 1 mg/ml; determined with column elution according to EU-Guideline RL67-548-EWG, annex V, chapter A6. The polymer-based film can, for example, comprise polymers such as polyurethane, polyether urethane, polyester urethane, polyester, polypropylene, polyethylene, polyamide, polyvinylchloride, polyorgano-siloxane (silicone), polyacrylate, polymethacrylate or mixtures therefrom, preferably polyurethane.

It is preferred that the polymer-based film exhibits a water vapor permeability from at least 300 g/m²/24 hrs, in particular from at least 1000 g/m²/24 hrs and especially from at least 2000 g/m²/24 hrs to 5000 g/m²/24 hrs, determined according to DIN EN 13726, upright.

In a preferred embodiment the assembly comprises a laminate according to the invention and a textile or a polymer-based film, wherein the laminate is arranged such that the first layer of the laminate is the wound contact layer. It is further preferred that the laminate comprises a second layer comprising a medically acceptable adhesive. The textile or the polymer-based film, preferably a polymer-film, is arranged on said second layer comprised by the present laminate.

In an alternatively preferred embodiment, the assembly comprises a laminate according to the invention, a textile or a polymer-based film and a layer comprising absorbent material. An absorbent material can be referred to as a material capable of receiving and subsequently retaining a liquid such as water or exudate. Absorbent materials are known in the art. In a preferred embodiment the absorbent material is a superabsorbent material based on a copolymer of acrylic acid and acrylamide or a foam, preferably a foam based on polyurethane. The layer of the absorbent material such as a foam can preferably have a surface being smaller that the surface of the second layer comprised by the laminate. It is further preferred that the layer of absorbent material is arranged on or adhered to the centre of the second layer comprised by the laminate, such that the four rims of the second layer are formed which are not covered by the layer of absorbent material and which have substantially of the same area size. In a preferred embodiment the textile or a polymer-based film can be adhered to the medically acceptable adhesive of said rims of the second layer comprised by the laminate. Put differently, the first layer of the present laminate can be used as wound contact layer, while the second layer comprised by the laminate enables the formation of an assembly optionally having further layers.

Moreover, the invention is related to a wound care product comprising the perforated adhesive laminate according to the invention and/or the assembly according to the invention. As far as the perforated adhesive laminate and/or the assembly are concerned, it applies substantially the same as described above. Preferably the wound care product is a wound dressing.

Further, the invention is directed to the use of the perforated adhesive laminate according to the invention as a wound contact layer or as a backing layer of a wound dressing. It is preferred that the first layer comprised by perforated adhesive laminate according to the invention is used as a wound contact layer of a wound dressing. The properties of the layer ensure an excellent adherence to the surface of the skin and enable a safe and easy removal without negatively affecting the surface of the skin or wound to which it has been applied.

In a preferred embodiment the perforated adhesive laminate according to the invention is used as a wound contact layer of an island-like wound dressing. As far as the first layer comprised by the laminate is concerned, the same as above applies. The island-like wound dressing further preferably comprises a laminate having a second layer comprising a medically acceptable adhesive on which a layer of absorbent material is adhered. With refence to the above the surface of the layer comprising the absorbent material is preferably smaller than the surface of the second layer comprised by the laminate and preferably is arranged on or adhered to the centre of the second layer comprised by the laminate, such that the four rims of the second layer are formed. These rims which are not covered by the layer of absorbent material preferably have substantially the same area size. In a preferred embodiment the textile or a polymer-based film can be adhered to the medically acceptable adhesive of said rims of the second layer comprised by the laminate. In other words, the first layer comprised by the present laminate can be used as the wound contact layer, while the second layer comprised by the laminate enables the formation of an assembly optionally having further layers.

In an alternative embodiment the perforated adhesive laminate according to the invention can be used as a backing layer of a wound dressing, in particular of a wound dressing having a pad, preferably a pad comprising an absorbent material, wherein the pad is directly into contact with the wound. It is preferred that the pad, preferably the pad comprising an absorbent material, is of such a size that the four rims of the first layer overlap the pad thus adhere to the surface, preferably the skin, surrounding the pad. It is further preferred that the perforated adhesive laminate does not comprise a second layer comprising a medically acceptable adhesive. In other words, the substrate of the perforated adhesive laminate can be considered as the backing layer of a wound dressing.

In an alternative embodiment the assembly according to the invention can be used as a backing layer of a wound dressing, in particular a wound dressing having a pad, preferably a pad comprising an absorbent material, wherein the pad is directly in contact with the wound. It is preferred that the pad, preferably the pad comprising an absorbent material, is of such a size that the four rims of the first layer overlap the pad to adhere to the surface, preferably the skin, surrounding the pad. It is further preferred that the assembly comprises a laminate comprising layer (a), (b) and (c) as well as and a textile or a polymer-based film, wherein the textile or a polymer-based film is arranged on or adhered to (c) the second layer comprising a medically acceptable adhesive. In other words, the textile or the polymer-based film of the assembly can be considered as a backing layer of a wound dressing.

### EXPERIMENTAL PART

### 1. Orientation of the Perforation Pattern

### Laminate:

Layer (a): Silicone adhesive with a thickness of 120 μm
Substrate (b) Polyurethane film with a thickness of 25 μm
Layer (c): Acrylic adhesive with a thickness of 30 μm

Four laminates of about 175×175 mm are perforated with holes having a diameter of 2 mm with a known perforation method, ultrasonic cutting, such that four different perforation patterns each having an area of the openings of the perforated holes of about 25% of the total surface of the laminate are obtained. These patterns are shown in FIGS. 1 to 4.

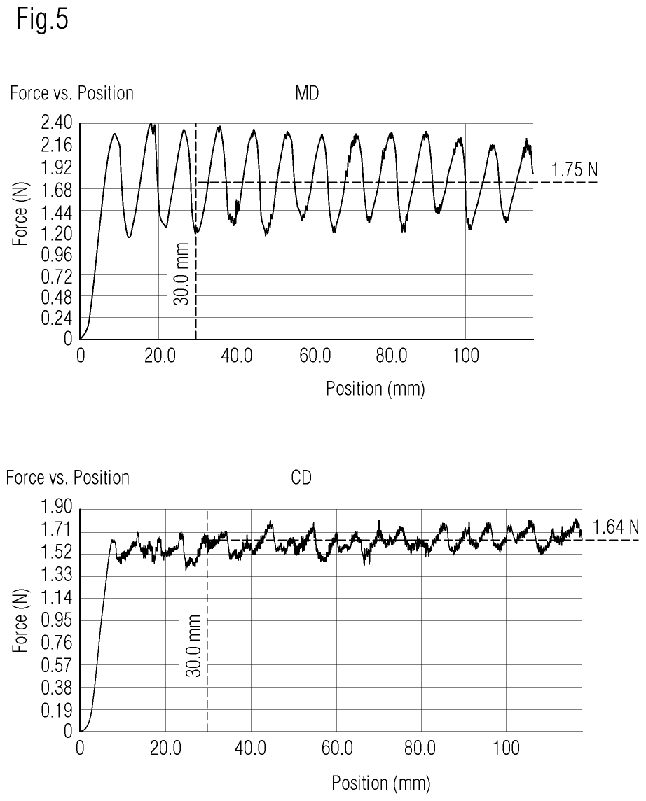

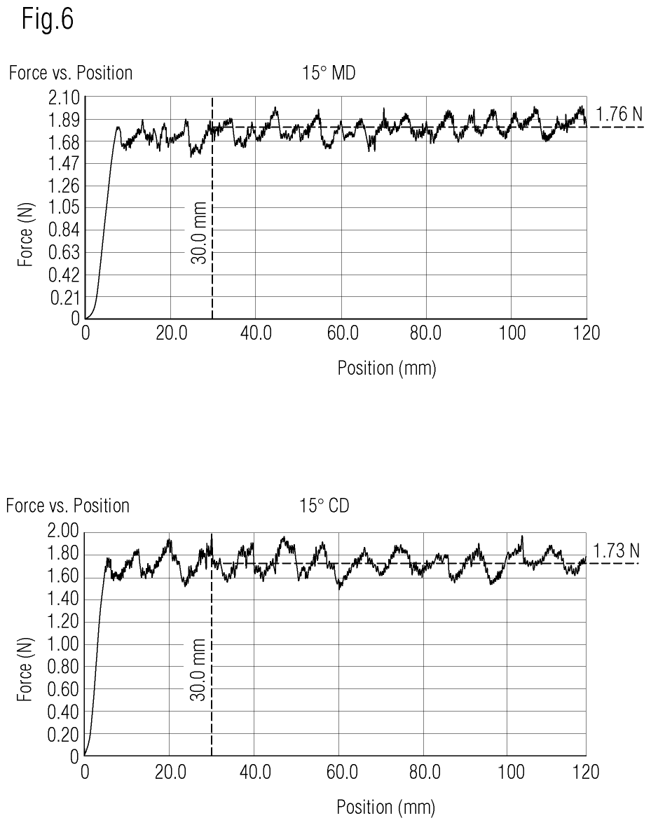

The peel force on Bristol paper of a laminate having a pattern according to FIG. 1 and a pattern according to FIG. 4 has been determined and is shown in FIGS. 5 and 6, wherein the peel force has been determined by a method based on FINAT n° 1. The peel force is determined over a width of 25 mm. The characteristic values are shown Table 1 (lines with 0° MD, 0° CD correspond to a pattern according to FIG. 1; lines with 15° MD, 15° CD correspond to a pattern according to FIG. 4).

TABLE 1

| | peaks | valleys | peel force | | amplitude/ |
|---|---|---|---|---|---|
| | average | average | average | amplitude of | peel force |
| tilt | [N] | [N] | [N] | peaks/valleys | [%] |
| 0° MD | 2.24 | 1.22 | 1.75 | 1.02 | 58 |
| 0° CD | 1.78 | 1.52 | 1.64 | 0.26 | 16 |
| — | — | — | — | — | — |
| 15° MD | 1.86 | 1.52 | 1.76 | 0.34 | 20 |
| 15° CD | 1.85 | 1.52 | 1.73 | 0.33 | 20 |

MD: machine direction
CD: cross direction

As can be seen from that table, a perforation pattern according to FIG. 4 (tilt)15° has a strength of average peel force in machine-direction and a strength of average peel force in cross-direction which differ less than 2%. Moreover, the amplitude between the maximum and minimum peaks of the peel force curve is 20% of the average peel force.

### 2. Preparation of a Perforated Laminate with Two Liners

On an unperforated laminate as described in Example 1, a polyethylene liner is applied to layer (a), i.e. a silicone adhesive with a thickness of 120 μm and a paper liner is applied to layer (c), i.e. an acrylic adhesive with a thickness of 30 μm. Subsequently, die cutting is carried out on a rotary die apparatus, wherein the rotary die cutting unit comprising a cylindrical rotary cutting tool (die) with a diameter of 2.4 mm, wherein the cylindrical rotary cutting tool (die) faces a counter flat cylinder.

The laminate to be die cut passes through both cylinders, wherein the polyethylene liner and the laminate are fully perforated but the paper liner is not. Such a perforation is also referred to as “kiss-cutting”. After the removal of the perforated polyethylene liner a new polyethylene liner is applied. Further, after the removal of the not fully perforated paper liner a new paper liner is applied.

### 3. Properties of an Assembly Comprising the Perforated Adhesive Laminate and a Polymer-Based Film

### Laminate:

l Layer (a): Silicone adhesive with a thickness of 150 μm
Substrate (b) Polyurethane film with a thickness of 25 μm
Layer (c): Acrylic adhesive with a thickness of 30 μm

Nine laminates of about 175×175 mm are perforated each with holes of a specific diameter with a known perforation method, ultrasonic cutting, such that each laminate has a perforation pattern according to FIG. 4. Subsequently a polyurethane film of 15 μm is adhered to the second layer (c) of each laminate. The characteristic parameters of the samples are shown in Table 2.

TABLE 2

| | Diameter of the | Number of | Open |
|---|---|---|---|
| | perforation hole | perforation holes | area |
| Sample | [μm] | per cm² | [%] |
| 1 | 1.3 | 12.4 | 16.5 |
| 2 | 1.4 | 12.4 | 19.1 |
| 3 | 1.5 | 12.4 | 21.9 |
| 4 | 1.6 | 12.4 | 25 |
| 5 | 1.7 | 12.4 | 28.2 |
| 6 | 1.7 | 11.0 | 25 |
| 7 | 1.8 | 9.8 | 25 |
| 8 | 1.9 | 8.8 | 25 |
| 9 | 2.0 | 7.9 | 25 |

Open area: sum of the areas of the openings of the perforated holes divided by the total surface of the laminate

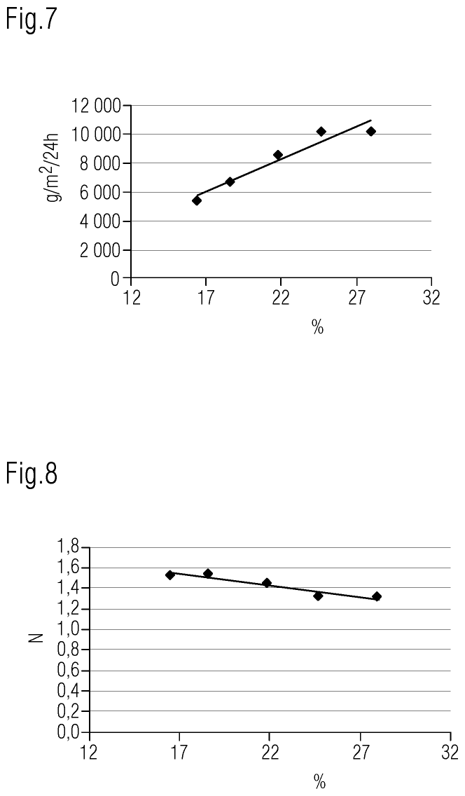

Further, the moisture vapor transmission rates (determined by DIN EN 13726-2:2002, upright) of samples 1, 2, 3, 4 and 5 has been determined. A curve showing the values of the moisture vapor transmission rates in “g/m²/24 h” (Y-axis) and the corresponding values of the open area in “%” (X-axis) has been prepared. The corresponding diagram is shown in FIG. 7.

Further, the average peel force of samples 1, 2, 3, 4 and 5 has been determined. A curve showing the values of the average peel force in “N” (Y-axis) and the corresponding values of the open area in % (X-axis) has been prepared. The corresponding diagram is shown in FIG. 8.

Peel force and the moisture vapor transmission rate are apparent antinomic properties. Moisture vapor transmission rate rises with an increasing open area while the peel force decreases with an increasing open area (see FIGS. 7 and 8).

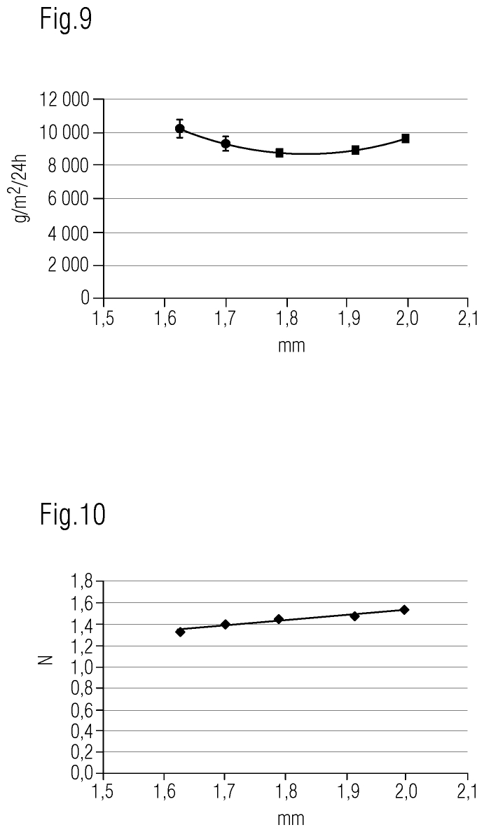

Further, the moisture vapor transmission rates (determined by DIN EN 13726-2:2002, upright) of samples 4, 6, 7, 8 and 9 (all having an open area of 25%) have been determined. A curve showing the values of the moisture vapor transmission rates in “g/m²/24 h” (Y-axis) and the corresponding values of the diameter of the opening of the perforation holes in “mm” (X-axis) has been prepared. The corresponding diagram is shown in FIG. 9.

Further, the average peel force of samples 4, 6, 7, 8 and 9 (all having an open area of %) has been determined. A curve showing the values of the average peel force in “N” (Y-axis) and the corresponding values of the diameter of the opening of the perforation holes in “mm” (X-axis) has been prepared. The corresponding diagram is shown in FIG. 10.

From FIGS. 9 and 10 it can be derived that, taking a fixed open area, the diameter of the perforation holes plays an important role for the corresponding average peel force and the moisture vapor transmission rate of the assembly. In summary, taken a fixed open area, bigger perforation holes would result in a good peel force as well as in a good moisture vapor transmission rate.

1-17. (canceled)

18. A perforated adhesive laminate comprising
(a) a first layer comprising a first medically acceptable adhesive,
(b) a substrate
(c) optionally a second layer comprising a second medically acceptable adhesive,
wherein the first layer is applied on one side of the substrate and wherein the optional second layer is applied on the opposite side of the substrate; and
wherein the laminate has perforation holes forming a perforation pattern such that the strength of the first layer's average peel force in machine-direction and the strength of the first layer's average peel force in cross-direction differ no more than 5% and/or the amplitude between the maximum and minimum peaks of the peel force curve are no more than 25% of the average peel force; and
wherein the openings of the perforation holes have an area of between 1.75 mm² to 12.5 mm².

19. The perforated adhesive laminate according to claim 18, wherein the first layer is a wound contact layer.

20. The perforated adhesive laminate according to claim 18, wherein the first adhesive comprised by the first layer is a silicon-based adhesive.

21. The perforated adhesive laminate according to claim 18, wherein the substrate is a film.

22. The perforated adhesive laminate according to claim 18, wherein the second adhesive comprised by the second layer is an acrylate-based adhesive.

23. The perforated adhesive laminate according to claim 18, wherein the openings of the perforated holes have an area between 2.25 mm² and 5.0 mm².

24. The perforated adhesive laminate according to claim 18, wherein the perforation holes are arranged at equidistance from each other to form a square aligned to machine direction and cross direction.

25. The perforated adhesive laminate according to claim 18, wherein the perforation holes are arranged at equidistance from each other to form a square with a 45° angle from machine direction and cross direction.

26. The perforated adhesive laminate according to claim 18, wherein the perforation holes are arranged at equidistance from each other to form an equilateral triangle, wherein aligned holes constitute an angle of between 10° and 20° from machine direction and cross direction.

27. The perforated adhesive laminate according to claim 18, wherein the sum of the areas of the openings of the perforated holes is between 10% and 50% of the total surface of the laminate.

28. The perforated adhesive laminate according to claim 18, wherein a liner is applied to the first layer comprising a medically acceptable adhesive.

29. The perforated adhesive laminate according to claim 18, comprising the second layer comprising the second medically acceptable adhesive, wherein the first layer is applied on one side of the substrate and wherein the second layer is applied on the opposite side of the

30. The perforated adhesive laminate according to claim 18, wherein the first medically acceptable adhesive is different than the second medically acceptable adhesive.

31. A method for the preparation of the perforated adhesive laminate according to claim 18 comprising the steps of:
(i) providing a laminate comprising (a) a first layer comprising a first medically acceptable adhesive, (b) a substrate, (c) optionally a second layer comprising a second medically acceptable adhesive,
wherein the first layer is applied on one side of the substrate and wherein the optional second layer is applied on the opposite side of the substrate, and
wherein optionally a liner is applied to the first and/or the second layer comprising a medically acceptable adhesive
(ii) die cutting the laminate provided by step (i), wherein die cutting is performed such that the perforation holes are arranged at equidistance from each other.

32. The method of claim 31, wherein the perforation holes are arranged at equidistance from each other to form an equilateral triangle, wherein aligned holes constitute an angle of between 10° and 20° from machine direction and cross direction.

33. An assembly comprising the perforated adhesive laminate according to claim 18 and a textile or a polymer-based film.

34. The assembly according to claim 33, wherein the textile is a woven or nonwoven fabric.

35. The assembly according to claim 33, wherein the polymer-based film is based on polyethylene, polypropylene, polyurethane or copolymers or blends thereof.

36. A wound care product comprising the perforated adhesive laminate according to claim 18.

37. The wound care product of claim 36, wherein the perforated adhesive laminate is a wound contact layer or backing layer of an island-like wound dressing.
